(19) United States
(12) Patent Application Publication
(10) Pub. No.: US 2018/0085397 A1
(43) Pub. Date: Mar. 29, 2018

(54) One day burns treatment discovery for an industrial material

(71) Applicant: Husam Hussein Aljanabi, Vienna (AT)

(72) Inventor: Husam Hussein Aljanabi, Vienna (AT)

(21) Appl. No.: 15/278,337

(22) Filed: Sep. 28, 2016

(51) Int. Cl. A61K 35/06

(52) U.S. Cl. CPC A61K 35/06

(57) ABSTRACT

The grease (Machines lubricant), as burns medicine (treatment for burns), which is consisting of: 70-95% Base oil, 3-30% Thickner, F0-10% Additives, which is preventing swelling and the pain after burns, when it is used from the first-Aid Kits. Furthermore, it is stopping the pain in short time. In addition, it is new and for the first time to use the grease in the medicial field, which is originally used in the industrial field.

The invention concerns a medical use for an existing industrial material as a medicine for skin burns.

This material is (the Grease-as shown in the attached figures—furthermore, i refer to FIG. 3).

I want to pay attention, that my invention is about treating burns by using the grease (refer tot he figures), not the grease itself (not like other patents, Which is about the grease itself—only two oft hem are about burns, which are based on pharmatical based and the other about tomato based treatment).

How the burns come, by exposing the body to heat greater than 120° F. (49° C.). The causes of the burns/heat may come from the sun (sun burn), hot liquids, steam, fire, electricity, friction (causing rug burns and rope burns), and chemicals (causing caustic bum upon contact). Signs of a burn are direct pain, localized redness and swelling (in later stage).

### Definition of Burns and Burns Types

Burns are classified by burn degree, based on the severity of the tissue damage. A first-degree burn causes redness and swelling in the outermost layers of skin (epidermis). A second-degree burn involves redness, swelling and blistering and the damage may extend beneath the epidermis to deeper layers of skin (dermis). A third-degree burn, also called a full-thickness burn, destroys the entire depth of skin, causing significant scarring. Damage also may extend to the underlying fat, muscle, or bone.

My treatment is only for the 1st degree burns and 2nd degree burns (in without blistering). The severity of the burn determines the type of treatment and also where the burned person should receive treatment. Minor burns may be treated at home (the workshop; in this case) or in a doctor's office. These are defined as first- or second-degree burns covering less than 15 percent of an adult's body. In our case and medicine, this treatment is for the (Thermal burns), because there are another kinds of burns, inwhich i didn't treat it, like (chemical burns and electrica burns).

For the following kinds of burns, i recommend to go to specialized burns unit in the nearest hospital; face burns, eyes burns, genitals burns and also, if the the area of burns were reached 15% or more, from the body skin area (estimated percentage).

### The Problem:

Firstly, my previous job (profession) was Blacksmith, which is a profession specialized in manufacturing steel structures & equipments. In our previous profession, we are dealing all the time with (steel welding instruments, and cutting Oxy Astelin torch), for that, these making the steel parts hot for long-time after any procedure. Unfortunately, the place and the workshop, is in a very big industrial zone, which is a far from hospitals and emergency portals. Moreover, there are not enough First Aid and safety procedures. Furthermore, the income is low, as well as, there is no active health insurance. As a result of all the aforementioned, all the time, we face several kinds of accidents, one of that, is the frequent burns.

By dealing almost the time with machines & big vehicles, which are containing almost in each joint, which connects two/one side movable metal parts, the grease, so we take it and covert he burnt spot by a layer, direct to cool the burn, which is available in these machines. By putting a layer from that sticky material on the burnt spot, it makes direct cold feeling, then i discovered the magical medical properties and the treatment ability of the (Greases see the attached figures). It makes the same effect, is it was used (from the machine) or new (not used).

Besides, we have tried before, to keep for emergency burns, creams or Ointments from pharmacies, but it weren't so sufficient to reduce the big pain after the burns. Even, in 70-90% from the burns, it doesn't prevent the swelling which comes after the burns (swell normally comes after the burns, in about few hours). I have used it personally many times and i can prove it infront of you, by making burn, then treat myself at the same day. It is thick and gelly material, which is helping for immidiate cooling and stopping the pain of the burned spot of the skin. In addition, it is not expensive and every one can keep it at home in the emergency kit (First Aid box). From its advantages, it is preventing the pain of the burns and prevent the bulge (swelling with liquid) which is associated with/or after burning, if it was used fast. Furthermore, it is reacting fast, one day treatment, then forget the pain or the swelling oft he burns.

### Composition of Grease:

The grease (Machines lubricant), most of it, are consisting of mineral oil as their fluid components, thickner and additives. These mineral oil-based greases: typically provide satisfactory performance in most industrial applications. In temperature extremes (low or high), a grease that utilizes a synthetic base oil will provide better stability. The Thickner: is a material that, in combination with the selected lubricant, will produce the solid to semifluid structure. The types of the thickners, For example; metalic soap and Non-metalice soap, the metalic soap include; lithium, aluminum, sodium and calcium. Lately, complex thickner type greases are gaining popularity.

Non-soap thickners are also gaining popularity in special applications such as high-temperature environments, such a (clay and polyurea). Bentonite and silica aerogel are two examples of thickners that don't melt at high temperatures. There is a misconception, however, that even though the thickner may be able to withstand the high temperatures, the base oil will oxidize quickly at elevated temperatures, thus requiring a frequent relube interval.

Additives: It can play several roles in the lubricating grease, these primarily include enhancing the existing desirable properties, suppressing the existing undesirable properties, and imparting new properties. The most common additives are oxidation and rust inhibitors, extreme pressure, antiwear and friction-reducing agents.
1. Oil and grease for lubrication
2. Composition and characteristics of grease
3. Base oil
4. Vapor deposition agent
5. Additives
6. Ingredients and characteristics of grease (Example Products)

### Conditions of Use:

1—Don't use it, if the skin was discovered or in the opened skin, (need more studies for the safety on the discovered skin burns).
2—Don't use it for the pregnant women, the women whom are doing breast feeding or for the children. (need more studies on the pregnant, breast feeding women and on the children).

### Method of Use:

After you got burnt, go to the First Aid kit and get the grease, then cover the burned spot/spots or areas by a thick layer from the grease, thickness from 1 mm to 8 mm. Then keep it from few hours till one day maximum. After you feel that the pain was totally gone, then clean it by a piece of cloths or tissues then wash it with any cleaning material (for ex. Soap).

1. Treatments for Burns, by using the (Machines grease-lubricant).

2. Treatment according to claim (1), for Burns pain by using the grease as a medicine.

3. Treatment according to one of the claim (1), which prevents swelling which follows the burns.

4. Medical use and discovery for the (machinery lubricant) grease according to the claim (1).

5. Medicine according to the claim (1) for burns with one day treatment, by using the (lubricant) grease.

6. Medical use for the machinery grease (which is for industrial use) according to the claim (1), which is available in the markets only for machines.
